(19) United States
(12) Patent Application Publication
(10) Pub. No.: US 2014/0147861 A1
(43) Pub. Date: May 29, 2014

(54) MARKERS TO IDENTIFY PRIMARY CELLS FROM TUMOR BIOPSIES

(71) Applicant: The Regents of the University of Califomia, Oakland, CA (US)

(72) Inventors: Edward L. Nelson, Irvine, CA (US); Trisha M. Westerhof, Irvine, CA (US)

(21) Appl. No.: 13/888,282

(22) Filed: May 6, 2013

Related U.S. Application Data
(60) Provisional application No. 61/643,141, filed on May 4, 2012.

(51) Int. Cl. G01N 33/574

(52) U.S. Cl. CPC G01N 33/574
USPC 435/7.23

(57) ABSTRACT

A process that utilizes a panel of primary monoclonal antibodies (mAbs) specific for cell markers that are directly conjugated to fluorophores (Alexa Fluor dyes and Quantum Dots). The mAb-fluorophore conjugates are used to interrogate the presence or absence and relative level of expression of each of the cell markers using laser scanning confocal microscopy. Complex tissues contain various cellular subsets each of which contribute in different ways to the biological behavior and each of which has, if selected well, a unique pattern of cell marker molecules capable of being identified by monoclonal antibodies and therefore provide a specific phenotype. The cell markers may be cell surface or intracellular in location. The expression patterns of heterogeneous mixtures of cells are detected by the mAb-fluorophore conjugates, and are used to decipher the identity of the cells based on their expression or lack thereof, of the cell markers.

The present application claims the benefit of and priority to U.S. Provisional Application No. 61/643,141 titled “MARKERS TO IDENTIFY PRIMARY CELLS FROM TUMOR BIOPSIES,” filed on May 4, 2012, which is hereby incorporated by reference in its entirety.

### FIELD

The embodiments provided herein relate generally to cell markers, and more particularly to systems and methods to identify cellular subsets within tumor biopsies via a multicolor immunofluorescent laser scanning confocal imaging strategy by interrogating cell markers, whose expression or lack thereof uniquely identifies particular cell subsets of interest, such as cancer stem cells, endothelial progenitor cells, as well as other primary adherent cells,

### BACKGROUND

The concept that tumors contain small cellular subsets, e.g. progenitor cells, which are associated with tumor biological behavior, has reframed many conceptual models upon which biological hypotheses are formulated. The proportion of these progenitor cells (i.e., cancer stem or tumor initiating cells, endothelial progenitor cells, etc.) within a tumor have been associated with biological behavior (e.g. growth, metastasis, angiogenesis, and therapeutic responses). Thus, enumerating the proportion of these cellular subsets is highly likely to impact our capacity to rationally design individualized therapeutic strategies to limit toxicity and optimize clinical benefit, a long-standing objective for cancer patients and clinicians. The cancer stem cell (CSC) model is being tested and refined, but many important biological questions remain unanswered, due in part to the challenges of identifying, recovering and studying unperturbed viable primary tumor cells such as CCs. For example, it remains to be determined if de novo isolated cancer stem cells exhibit the same sensitivity to cytotoxic agents as the majority of the malignant epithelium although it has been proposed that they are relatively resistant. Also, increased endothelial progenitor cells have been associated with an angiogenic phenotype and may be indicative of an increased response to anti-angiogenic therapy.

The amount of cell surface markers able to be identified in a single sample using immunofluorescent cell imaging has traditionally been limited by the fluorophores chosen to label each cell surface marker. Many fluorophores are commercially available for use, but their emission spectra often significantly overlap with one another such that it is difficult to confidently distinguish between the emissions of each individual fluorophore. There have been algorithms implemented into the imaging software provided by Zeiss and other microscopy companies called emission fingerprinting that can spectrally separate fluorophores whose emission spectra overlap, but this requires several control samples to be prepared and imaged each time the algorithm is to be used in order to calibrate the system. Such methods are not feasible for analyzing primary cells obtained from human tumor specimens or other primary tissue samples, because of the large quantity of cells necessary to include all the appropriate controls for emission fingerprinting.

An increasingly widespread requirement in biomedical research is the positive selection of single cells from populations of cells. Biologists have an increasing number and variety of tools with which to analyze single cells at the molecular level, including microscopy, PCR, patch-clamp, and microanalytical chemical separations, yet have very limited tools to enable the selection of a single cell from a large cellular population.

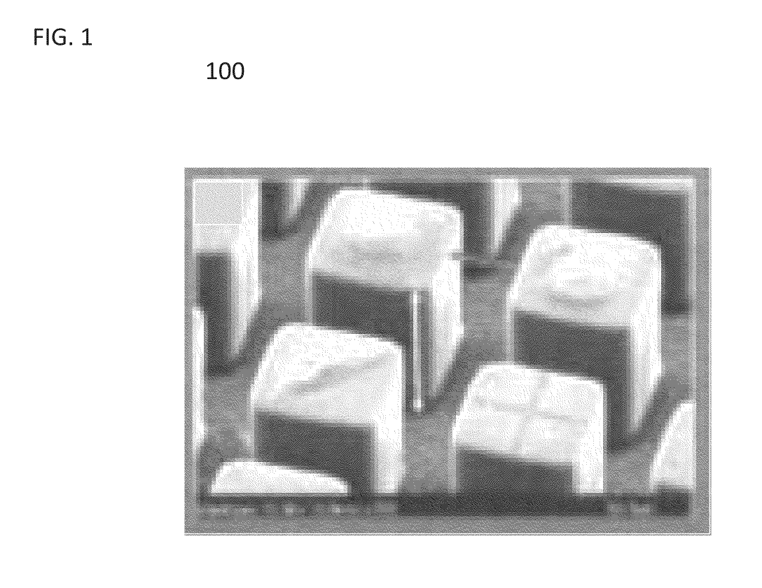

FIG. 1 illustrates an SEM micrograph 100 of a micropallet array. Micropallet arrays, the fabrication of which has been previously disclosed in U.S. Published Patent Application No. 2007-0292312, are composed of many individual, releasable polymer pedestals (micropallets) that isolate adherent cells in culture. They can be released from the substrate (glass or otherwise) using a laser or otherwise. Magnetically responsive, ferromagnetic micropallets, which have been previously disclosed in U.S. Pat. Nos. 7,659,954 and 7,951,580, are collectable using magnetically-based systems. In use, the micropallets or microstructures can be released from the substrate by any means including, but not limited to, laser-based release or direct-contact mechanical disruption of the attachment to the underlying substrate, after which they are collected using a magnetic collection probe based on magnets.

Once released, the individual micropallets are collectable such that any cell or cells adhered to a single micropallet can be isolated, selected, and collected from a larger cellular population. The process has been shown to cause minimal perturbation to the released and recovered cells and the cells remain viable and capable of expanding into clonal populations (derived from a single cell) after recovery.

While this system is appropriate for collecting single cell samples within pure populations of a single cell type, the user is unable to effectively apply this collection strategy to a heterogeneous cell population plated on a micropallet array, because of the platform's inability to uniquely identify cell types within mixed populations of cells.

Although several strategies exist to selectively identify and collect cells from mixed populations, each has significant drawbacks for the analysis of primary adherent tumor cells. Fluorescence-activated cell sorting (FACS) requires large numbers of cells that have been subjected to enzymatic or mechanical tissue disruption. Laser capture microdissection (LCM) can collect single cells or small groups of selected cells or (non-viable) from fixed or frozen tissue sections. Live cell LCM protocols have been reported, but are uniformly inefficient, low throughput and not suitable for isolating significant numbers of single, living cells. Recently, live cell microarray technologies based upon ligand-receptor interactions have been reported, but these are critically dependent upon single, unique discriminating interactions for each cell type. These technologies are not readily applicable to the identification and recovery of rare cells that require multi-parameter detection and are poorly suited to evaluating multiple discrete cell populations within a complex sample. Although multicolor imaging, albeit not six channel, has been used extensively in histologic sections and whole mount preparations to identify cells expressing one or more markers, as noted above, these strategies do not permit the recovery of single cells in a manner amenable to analyses of cells from several subsets from a single sample.

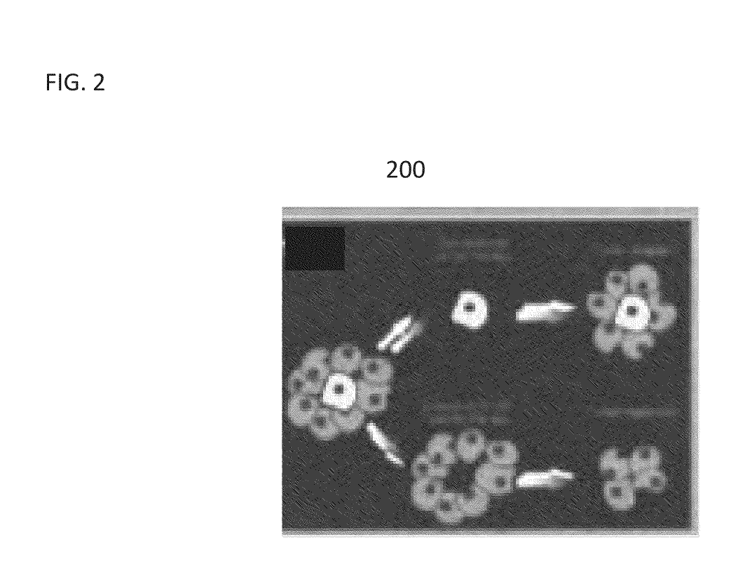

FIG. 2 illustrates a cancer stem cell hypothesis. Breast cancer patients, even those with histologically identical tumors, experience substantial variability in clinical behavior and response to treatment. Differences in the profile of tumors, such as the proportion of cancer stem cells (CSCs), may be a source of this variability (see FIG. 2 200). Cell subsets of interest can be identified by their expression of a panel of cell surface markers. In some applications, for example, the cancer stem cell hypothesis, the ability to analyze these rare cell subsets could lead to the development of more efficacious cancer therapies that directly target these cells.

### SUMMARY

Systems and methods utilizing markers to identify primary cells from tumor biopsies are disclosed. According to one embodiment, a cell identification strategy involves detecting the expression patterns of markers that are expressed on cells to uniquely identify different cell populations within heterogeneous mixtures of cells. The cell markers may be on the cell surface or intracellular in location. The cell markers are interrogated using monoclonal antibodies (mAbs) that are directly conjugated to flourophores. The mAb-flourophore conjugates are used to detect the presence or absence and relative level of expression of each of the cell markers using laser scanning confocal microscopy.

In another embodiment, a kit for identifying cells is provided that includes first and second groups of mAb-fluorophores. The first and second groups of mAb-fluorophores comprising differing cell markers identifiable by differing fluorophore emission spectra.

The systems, methods, features and advantages of the invention will be or will become apparent to one with skill in the art upon examination of the following figures and detailed description. It is intended that all such additional methods, features and advantages be included within this description, be within the scope of the invention, and be protected by the accompanying claims. It is also intended that the invention is not limited to require the details of the example embodiments.

### BRIEF DESCRIPTION

The details of the embodiments provided herein, including fabrication, structure and operation, may be gleaned in part by study of the accompanying figures, in which like reference numerals refer to like parts. The components in the figures are not necessarily to scale, emphasis instead being placed upon illustrating the principles of the invention. Moreover, all illustrations are intended to convey concepts, where relative sizes, shapes and other detailed attributes may be illustrated schematically rather than literally or precisely.

FIG. 1 illustrates an SEM micrograph of a micropallet array.

FIG. 2 illustrates a cancer stem cell hypothesis.

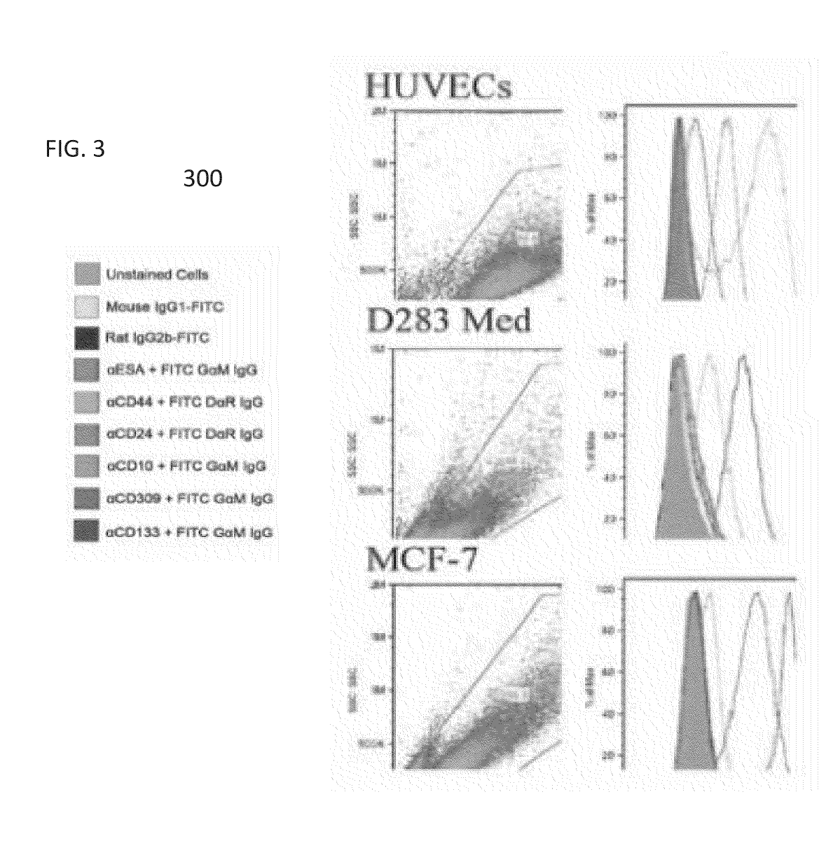

FIG. 3 illustrates an exemplary multicolor imaging strategy, according to one embodiment.

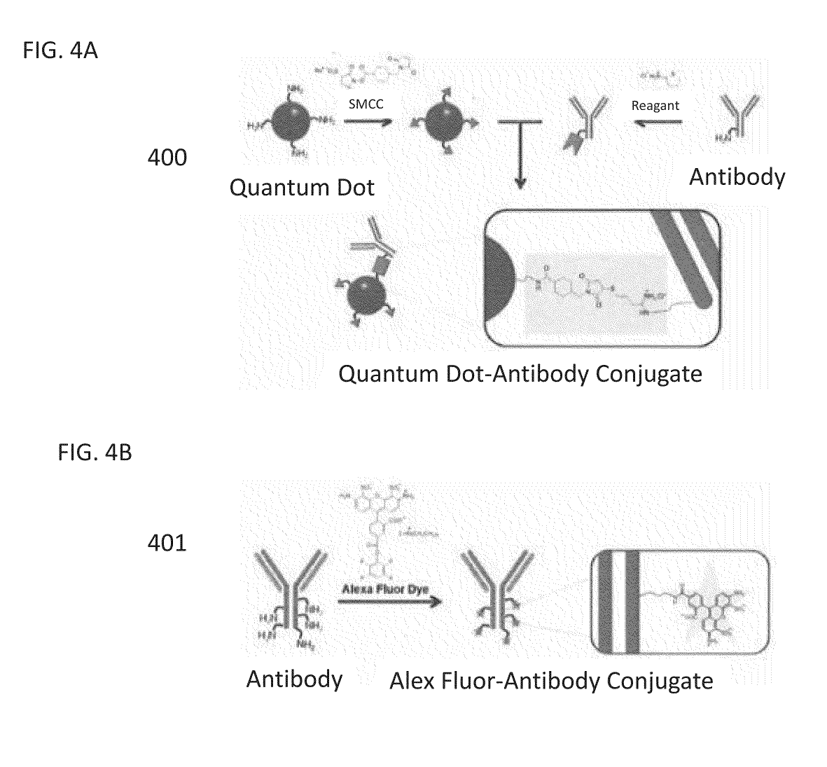

FIG. 4A illustrates an exemplary Qdot-mAb conjugation scheme used to couple monoclonal antibodies to respective fluorphores.

FIG. 4B illustrates an exemplary Alexa Fluor-mAb conjugation scheme used to couple monoclonal antibodies to respective fluorphores.

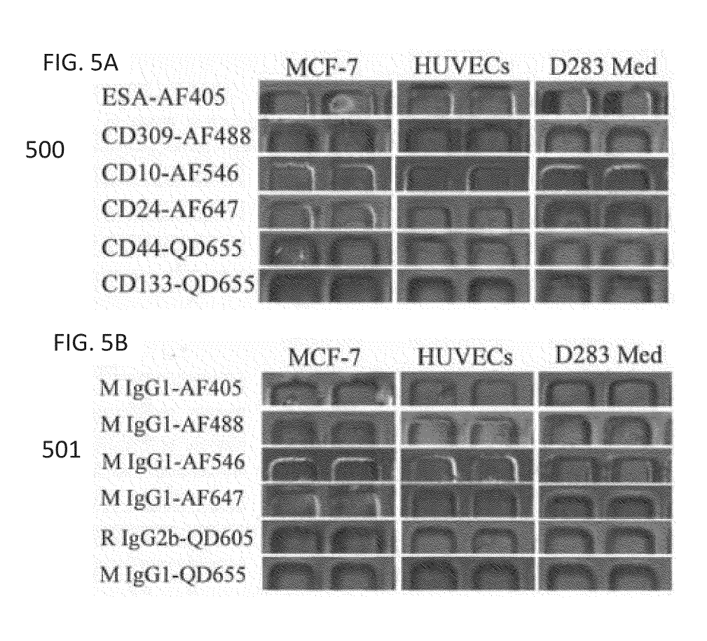

FIG. 5A illustrates exemplary immunoflourescent detection of single cell surface markers using fluorophore conjugated monoclonal antibodies, according to one embodiment.

FIG. 5B illustrates exemplary conjugation of mouse IgG1 and rat IgG2b isotype antibodies to respective dyes as controls, according to one embodiment.

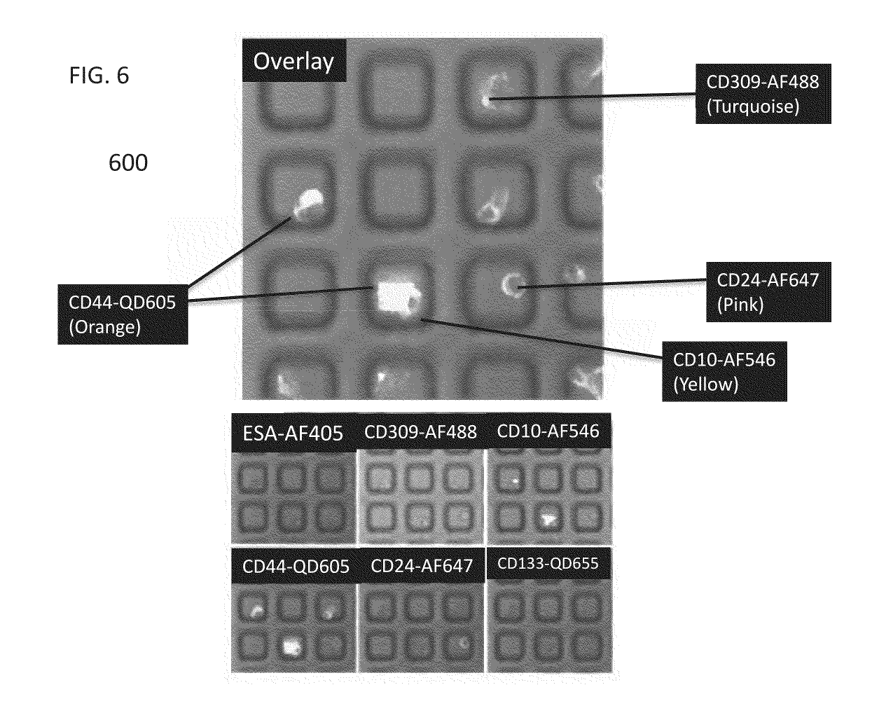

FIG. 6 illustrates exemplary multicolor immunoflourescent detection of cell surface markers, according to one embodiment.

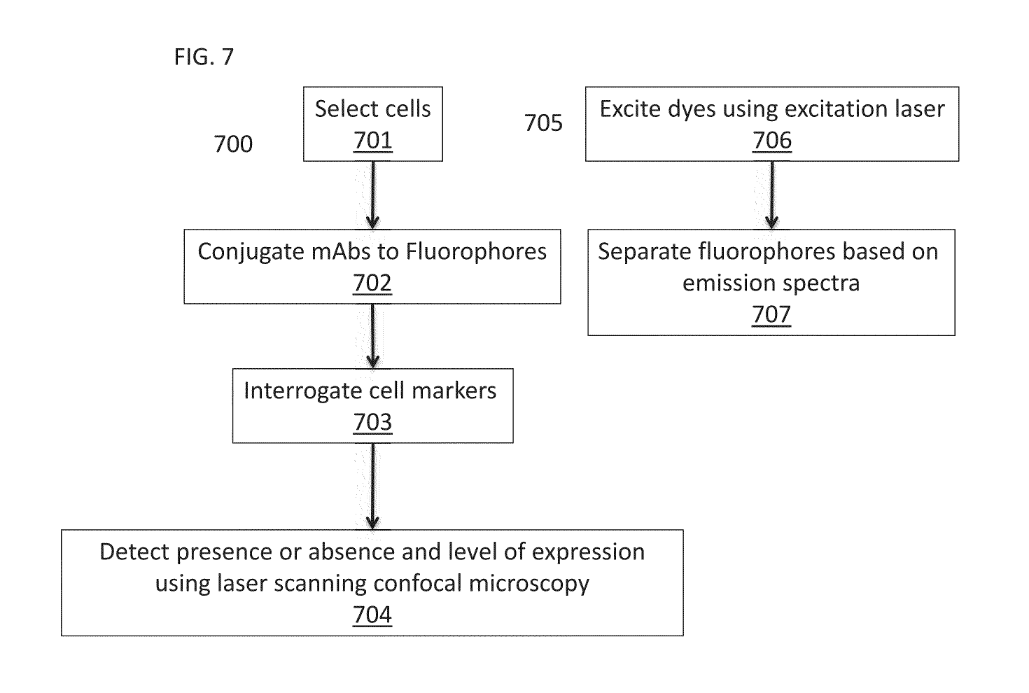

FIG. 7 illustrates an exemplary cell identification strategy for use with the present system, according to one embodiment.

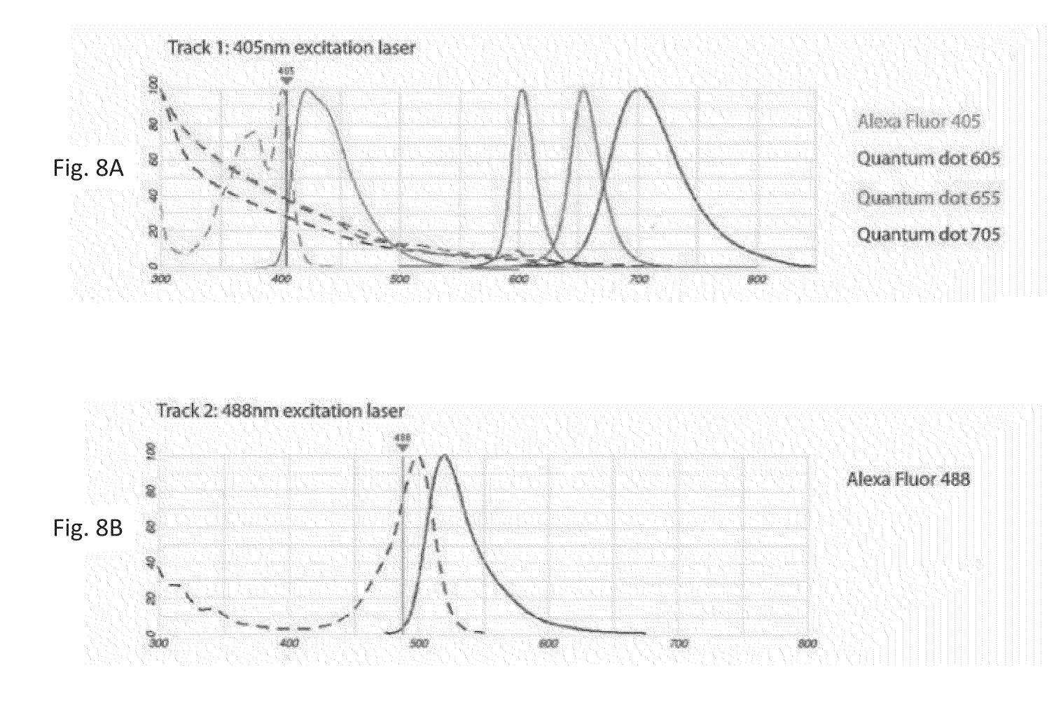

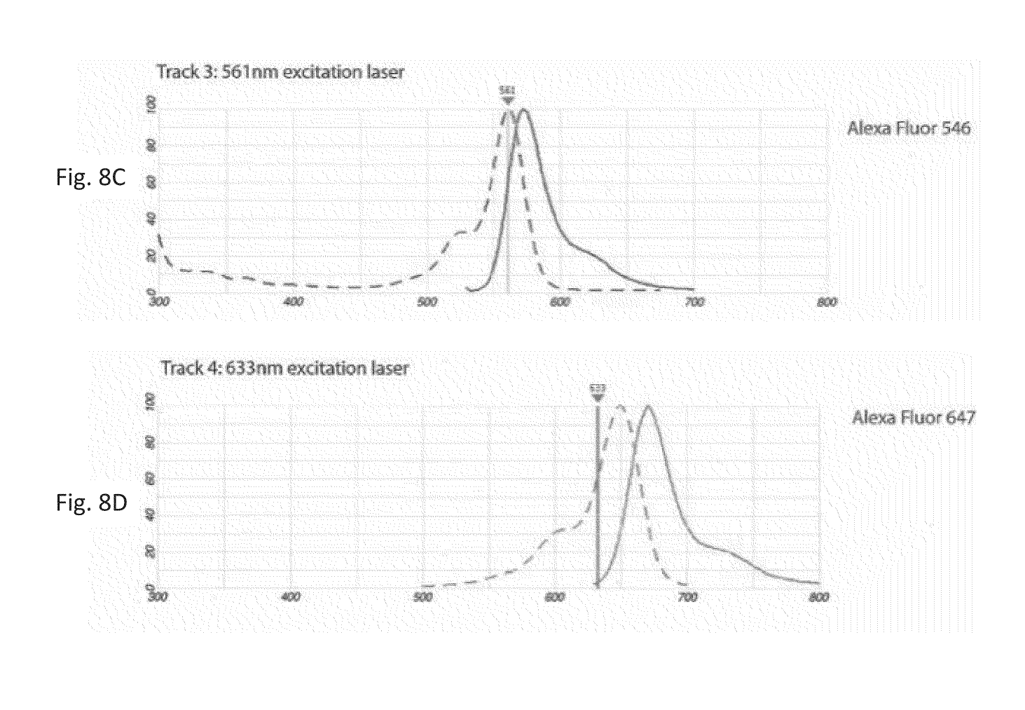

FIGS. 8A-D illustrate emission spectra resulting from a multicolor imaging strategy of fluorophores, according to one embodiment.

It should be noted that the figures are not necessarily drawn to scale and that elements of similar structures or functions are generally represented by like reference numerals for illustrative purposes throughout the figures. It also should be noted that the figures are only intended to facilitate the description of the various embodiments described herein. The figures do not necessarily describe every aspect of the teachings disclosed herein and do not limit the scope of the claims.

### DETAILED DESCRIPTION

The systems and methods described herein are directed to a process that utilizes a panel of primary monoclonal antibodies (mAbs) specific for cell markers that are directly conjugated to fluorophores (Alexa Fluor dyes and Quantum Dots). The mAb-fluorophore conjugates are used to interrogate the presence or absence and relative level of expression of each of the cell markers using laser scanning confocal microscopy. Complex tissues contain various cellular subsets each of which contribute in different ways to the biological behavior and each of which has, if selected well, a unique pattern of cell marker molecules capable of being identified by monoclonal antibodies and therefore provide a specific phenotype. The cell markers may be cell surface or intracellular in location. However, the technology to identify, enumerate, isolate, recover and analyze, with minimal perturbation, uniquely identified adherent cells from one or more cellular subsets out of a heterogeneous mixture or complex tissue, does not currently exist. The expression patterns of heterogeneous mixtures of cells are detected by the mAb-fluorophore conjugates, and are used to decipher the identity of the cells based on their expression or lack thereof, of the cell markers. By using predetermined combinations of mAb-fluorophores, it is possible to identify and differentiate between a variety of cell subsets that comprise complex tissues, for example, neoplastic tumors. For exemplary purposes only, the present disclosure focuses on human breast tumors and cellular subsets, including putative cancer stem cells, endothelial progenitor cells, myoepithelial cells, epithelial tumor cells, among others.

The embodiments described herein have the ability to multiplex the detection of several cell surface markers within a given sample because the emission spectra of the selected fluorophores do not significantly overlap with one another, or are excited with different excitation lasers such that their emissions do not interfere with one another. This ability, along with the use of the devised panel of cell surface markers, enables the user to identify several cell populations within a single sample without the need for emission fingerprinting. This approach has yielded an innovative methodology that 1) permits simultaneous enumeration of various 3 cellular elements present within a complex adherent cell sample, 2) provides the opportunity to assess the selected molecular profiles of individual cells from these various cellular compartments, 3) can be available to patients at the time of diagnosis vs. after tumor resection, 4) overcomes limitations to existing technologies, such as laser micro-dissection, and 5) is designed for high throughput analyses.

The embodiments described herein are directed to the development of a multicolor immunofluorescent imaging strategy that enables the user to uniquely identify and enumerate primary human cells with defined phenotypes from complex tumor samples. Beyond providing a novel tool for addressing fundamental biological questions in complex tissues or tumors, the embodiments described herein are positioned to be able to direct individualized treatment strategies for cancer patients at the time of diagnosis, with rapid turn around time, and little to any additional risk. One can also easily envision this platform being applied to the evaluation of other non-neoplastic complex tissues either at risk for or experiencing pathophysiologic processes. The embodiments described herein provide a combination of commercially available fluorophores that would not require emission fingerprinting in order to image multiple distinct fluorophores in a single sample.

TABLE 1
Cell surface marker expression patterns
of human breast tumor cell subsets.

| | ESA | CD44 | CD10 | CD24 | CD133 | CD309 | CD34 |
|---|---|---|---|---|---|---|---|
| Epithelial | + | + | − | + | − | − | − |
| Tumor Cells | | | | | | | |
| Mammary Tumor | + | + | − | − | − | − | − |
| Stem Cells | | | | | | | |
| Myoepithelial | − | +/− | + | − | − | − | − |
| Cells | | | | | | | |
| Endothelial | − | − | − | + | + | + | + |
| Progenitor | | | | | | | |
| Cells | | | | | | | |

TABLE 2
Control cell lines used to develop the breast
tumor cell subset identification strategy.

| | ESA | CD44 | CD10 | CD24 | CD133 | CD309 | CD34 |
|---|---|---|---|---|---|---|---|
| MCF-7 | + | + | − | + | *+/− | − | + |
| HUVECs | − | + | + | − | − | + | − |
| D283-Med | − | + | − | − | + | − | − |
| KG-1a | − | + | − | − | − | − | + |

*A small subset of MCF-7 cells have been reported to express CD133 and exhibit cancer stem cell properties.

TABLE 3
Human pancreatic tumor cell subsets.

| | ESA | CD44 | CD10 | CD24 | CD133 | CD309 | CD34 |
|---|---|---|---|---|---|---|---|
| Pancreatic | − | − | +/− | − | + | − | + |
| Tumor Stem | | | (SS) | | | | |
| Cells - Hermann | | | | | | | |
| Pancreatic | + | + | +/− | + | − | − | ?+/ − |
| Tumor Stem | | | (SS)* | | | | (SS)* |
| Cells - Li | | | | | | | |
| Pancreatic | + | + | − | − | − | − | − |
| Epithelial | | | | | | | |
| Tumor Cells | | | | | | | |
| Stromal Cells | − | − | − | − | − | − | +/− |
| | | | | | | | (SS) |
| Endothelial | − | − | − | + | + | + | + |
| Progenitor | | | | | | | |
| Cells | | | | | | | |

+/− (SS): expressed in a subset; +/− (SS)*: expressed in 14% overlapping population with Hermann phenotype.

TABLE 4
Control cell lines used to develop the pancreatic
tumor cell subset identification strategy.

| | ESA | CD44 | CD184 | CD24 | CD133 | CD309 | CD34 |
|---|---|---|---|---|---|---|---|
| MCF-7 | + | + | − | + | *+/− | − | − |
| HUVECs | − | + | +/− | − | − | + | − |
| D283-Med | − | + | − | − | + | − | − |
| KG-1a | − | + | − | − | − | − | + |
| HeLa | − | + | + | + | − | − | − |

The expression or lack thereof of the cell surface markers shown herein uniquely identify human breast tumor cell subsets of interest (Table 1). A total of 4 cell lines, MCF-7, Human Umbilical Vein Endothelia I Cells (HUVECs), KG-Ia, and D283 Med were selected based on their expression of the desired cell surface markers (Table 2). Additionally, the same rationale is applied for the identification of human pancreatic tumor cell subsets (Table 3), and its according control cell lines, MCF-7, HUVEC s, D283 Med, KG-Ia, and HeLa (Table 4). The cell lines collectively express the panel of surface markers and were used as controls to demonstrate proof of principle.

As an example, the panel of mAbs used to pair with the fluorophores has been formulated to identify various cell populations residing within a tumor including: epithelial tumor cells, mammary tumor stem cells, myoepithelial cells, and endothelial progenitor cells. For the individual cell populations the user is interested in identifying, there is no single marker that uniquely identifies one cell type from another, necessitating the need for a panel of surface markers in order to uniquely identify the cell type. There have been reports of cell surface marker panels in order to identify one of the cell types we are interested in identifying, but there are no reports of the multiplexed ability to identify all the individual cell populations within a given sample like the panel of cell surface markers we have devised and no reports of application of these markers to primary biopsy specimens. mAbs specific for each cell surface marker of interest were directly conjugated to Alexa Fluors and Quantum dots. In order to detect the expression of all the markers in a single sample, each fluorophore was paired with a mAb, such that the brightest fluorophores were paired with mAbs specific for lowly expressed surface markers and vice versa (Tables 5 and 6).

TABLE 5
mAb-Fluorophore conjugates for breast
tumor cell surface marker panel.

| mAb | Fluorophore |
|---|---|
| ESA | Alexa Fluor 405 |
| CD44 | Quantum Dot 605 |
| CD10 | Alexa Fluor 546 |
| CD24 | Alexa Fluor 647 |
| CD133 | Quantum Dot 655 |
| CD309 | Alexa Fluor 488 |
| CD34 | Quantum Dot 705 |

TABLE 6
mAb-Fluorphore conjugates for pancreatic
tumor cell surface marker panel.

| mAb | Fluorophore |
|---|---|
| ESA | Alexa Fluor 405 |
| CD44 | Quantum Dot 605 |
| CD184 | Alexa Fluor 546 |
| CD24 | Alexa Fluor 647 |
| CD133 | Quantum Dot 655 |
| CD309 | Alexa Fluor 488 |
| CD34 | Quantum Dot 705 |

The mAb-fluorophore conjugation chemistries are already established in literature. However, the mAb-fluorophore conjugates used in this panel are not readily available for purchase, and so the purified forms of each mAb had to be purchased and individually conjugated to each fluorophore. Covalent fluorophore-conjugated mAbs were utilized to minimize the possibility of cross-reactivity and non-specific binding of secondary reagents within a given sample. The mAbs were conjugated directly to Alexa Fluor dyes (Table 1) and Quantum dots (Table 2) using established crosslinking chemistries.

FIG. 3 illustrates an exemplary multicolor imaging strategy, according to one embodiment. The present system provides the ability to multiplex the detection of several cell surface markers within a given sample because the emission spectra of the selected fluorophores do not significantly overlap with one another, or are excited with different excitation lasers such that their emissions do not interfere with one another. This ability, along with the use of the devised panel of cell surface markers, enables the user to identify several cell populations within a single sample without the need for emission fingerprinting. To image the conjugates using laser scanning confocal imaging, the following multicolor imaging strategy was utilized (FIGS. 7, 8A-D). The 2 Quantum dots (Qdot 605 and 655) along with Alexa Fluor 405 are all excited 706 by the same ultraviolet excitation laser (405 m), and so were all grouped into a single imaging track. The remaining Alexa Fluor dyes (488, 546, and 647) are all excited by distinct wavelengths of light, and so each were divided into their own imaging track based on the laser used to excite each dye (488, 561, and 633 nm lasers, respectively). Based on the emission spectra illustrated in FIG. 8A, each fluorophore can be spectrally separated 707. The emission spectra of all 3 fluorophores excited by the 405 nrn laser do not overlap with one another, and each Alexa Fluor dye thereafter is excited separately, and so the fluorescence detected from each fluorophore can easily be distinguished from one another.

FIG. 4A illustrates an exemplary Qdot-mAb conjugation scheme used to couple monoclonal antibodies to respective fluorophores. FIG. 4B illustrates an exemplary Alexa Fluor-mAb conjugation scheme used to couple monoclonal antibodies to respective fluorphores. To interrogate the surface markers using laser scanning confocal microscopy, the flow cytometry validated purified mAbs were conjugated directly to Quantum dots (QD 400, FIG. 4A), and AlexaFluor dyes (AF 401, FIG. 4B) using established cross-linking chemistries. Covalent fluorophore-conjugated mAbs (primary labeling) vs. the use of secondary fluorophore reagents minimized the possibility of cross-reactivity and non-specific binding of secondary reagents within a given sample. In order to detect the expression of all the markers in a single sample, each fluorophore was paired with a mAb such that the brightest fluorophores were paired with mAbs specific for lowly expressed surface markers and vice versa. The resulting mAb-fluorophores were then used to stain and image each cell line (see FIG. 5A 500). Isotype antibodies were also conjugated to each fluorophore for appropriate negative controls (see FIG. 5B 501). FIG. 5A illustrates exemplary immunoflourescent detection of single cell surface markers using fluorophore conjugated monoclonal antibodies, according to one embodiment. FIG. 5B illustrates exemplary conjugation of mouse IgG1 and rat IgG2b isotype antibodies to respective dyes as controls, according to one embodiment.

Mixtures of the 3 cell lines were also stained and imaged to demonstrate the identification of each cell population based on their expression pattern of the 6 cell surface markers (see FIG. 6 600). FIG. 6 illustrates exemplary multicolor immunoflourescent detection of cell surface markers, according to one embodiment.

FIG. 7 illustrates an exemplary cell identification strategy 700 for use with the present system, according to one embodiment. The cell identification strategy 700 includes detecting the expression patterns of markers that are expressed on cells to uniquely identify different cell populations within heterogeneous mixtures of cells. Cells are selected based on their expression of desired cell surface markers 701. The cell markers may be on the cell surface or intracellular in location. The cell markers are interrogated 703 using the monoclonal antibodies (mAbs) that are directly conjugated 702 to fluorophores (the mAb-fluorophores). The mAb-fluorophore conjugates are used to detect the presence or absence and relative level of expression of each of the cell markers using laser scanning confocal microscopy 704. By using predetermined combinations of mAb-fluorophores, it is possible to identify and differentiate between a variety of cell subsets that comprise complex tissues, for example neoplastic tumors.

FIGS. 8A-D illustrate emission spectra resulting from a multicolor imaging strategy of fluorophores, according to one embodiment. The fluorophores were imaged using 4 imaging tracks that were separated by excitation laser (vertical lines at 405, 488, 561, and 633 nm). Excitation spectra (dashed lines) and the according emission spectra (solid lines) are as depicted on the graphs of wave length of light (nm) vs. % fluorescence intensity.

This approach has yielded an innovative methodology that 1) permits simultaneous enumeration of various cellular elements present within a complex adherent cell sample, 2) provides the opportunity to assess the selected molecular profiles of individual cells from these various cellular compartments, 3) can be available to patients at the time of diagnosis vs. after tumor resection, 4) overcomes limitation s to existing technologies, such as laser micro-dissection, and 5) is designed for high throughput analyses.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the spirit and scope of the appended claims.

Markers to identify primary cells from tumor biopsies have been disclosed. It is understood that the embodiments described herein are for the purpose of elucidation and should not be considered limiting the subject matter of the disclosure. Various modifications, uses, substitutions, combinations, improvements, methods of productions without departing from the scope or spirit of the present invention would be evident to a person skilled in the art.

What is claimed is:

1. A method for identifying cells, comprising:
exciting a plurality of fluorophores using one or more excitation lasers to produce emission spectra from the plurality of fluorophores;
spectrally separating the plurality of fluorophores into a plurality of groups of fluorophores based on the emission spectra;
conjugating a plurality of monoclonal antibodies (mAbs) having a predetermined cell surface maker to individual ones of the plurality of groups of fluorophores to produce a plurality of groups of mAb-fluorophores, wherein individual ones of the plurality of groups of mAb-fluorophores having an individual cell surface marker identifiable by an individual fluorophore emission spectra;
staining a plurality of cells with the plurality of groups of mAb-fluorophores; and
identifying individual ones of the plurality of cells based on an individual fluorophore emission spectra corresponding to an individual cell surface marker.

2. The method of claim 1, wherein the step of identifying comprises detecting the presence or absence of the cell surface marker and relative level of expression of the cell surface marker.

3. The method of claim 2, wherein the step of detecting comprises interrogating the plurality of cells using laser scanning confocal microscopy.

4. The method of claim 2, wherein the detected presence, absence, and relative level of expression of the cell marker are used to identify the cells.

5. The method of claim 1, wherein the plurality of cells comprise tumor cells.

6. The method of claim 1, wherein the fluorophores are selected from the group consisting of Alexa Fluors and Quantum Dots.

7. The method of claim 1, wherein the mAbs are selected from the group consisting of ESA, CD44, CD10, CD24, CD133, CD34, CD184, and CD309.

8. The method of claim 1, further comprising selecting cells for identification by obtaining a biopsy of a tumor.

9. The method of claim 8, wherein the tumor is one of breast or pancreatic.

10. The method of claim 1, wherein the step of conjugating monoclonal antibodies (mAbs) to fluorophores comprises pairing fluorophores having the brightest spectral emission with mAbs specific for lowly expressed surface markers.

11. A method for identifying cells, comprising:
staining a plurality of cells with the plurality of groups of mAb-fluorophores, wherein individual ones of the plurality of groups of mAb-fluorophores having an individual cell surface marker identifiable by an individual fluorophore emission spectra; and
identifying individual ones of the plurality of cells based on an individual fluorophore emission spectra corresponding to an individual cell surface marker.

12. The method of claim 11, wherein the step of identifying comprises detecting the presence or absence of the cell surface marker and relative level of expression of the cell surface marker.

13. The method of claim 12, wherein the step of detecting comprises interrogating the plurality of cells using laser scanning confocal microscopy.

14. The method of claim 12, wherein the detected presence, absence, and relative level of expression of the cell marker are used to identify the cells.

15. The method of claim 11, wherein the plurality of cells comprise tumor cells.

16. The method of claim 11, wherein the fluorophores are selected from the group consisting of Alexa Fluors and Quantum Dots.

17. The method of claim 11, wherein the mAbs are selected from the group consisting of ESA, CD44, CD10, CD24, CD133, CD34, CD184, and CD309.

18. The method of claim 11, further comprising selecting cells for identification by obtaining a biopsy of a tumor.

19. The method of claim 18, wherein the tumor is one of breast or pancreatic.

20. A kit for identifying cells, comprising:
a first plurality of mAb-fluorophores, wherein individual ones of the first plurality of mAb-fluorophores having a first cell surface marker identifiable by a first fluorophore emission spectra; and
a second plurality of mAb-fluorophores, wherein individual ones of the second plurality of mAb-fluorophores having a second cell surface marker identifiable by a second fluorophore emission spectra, wherein the second fluorophore emission spectra differs from the first fluorophore emission spectra.

21. The kit of claim 20, wherein individual ones of the a first plurality of mAb-fluorophores comprise a monoclonal antibody (mAbs) having the first predetermined cell surface maker conjugated with a fluorophore having the first fluorophore emission spectra when excited by an excitation laser, and wherein individual ones of the second plurality of mAb-fluorophores comprise a monoclonal antibody (mAbs) having a second predetermined cell surface maker conjugated with a fluorophore having the second fluorophore emission spectra when excited by an excitation lasers.

22. The kit of claim 21, wherein fluorophores of the first and second plurality of mAb-fluorophores are selected from a group consisting of Alexa Fluors and Quantum Dots.

23. The method of claim 21, wherein mAbs of the first and second plurality of mAb-fluorophores are selected from the group consisting of ESA, CD44, CD10, CD24, CD133, CD34, CD184, and CD309.
